(19) United States
(12) Patent Application Publication
(10) Pub. No.: US 2025/0319293 A1
(43) Pub. Date: Oct. 16, 2025

(54) EXTRUSION METHODS, EXTRUDED COMPOSITIONS, AND SYSTEMS THEREOF

(71) Applicant: University of Dayton, Dayton, OH (US)

(72) Inventors: Russell Kirk Pirlo, Oakwood, OH (US); Trevor Tilly, Sugarcreek Township, OH (US); Carter McCrea, Dayton, OH (US)

(21) Appl. No.: 19/175,151

(22) Filed: Apr. 10, 2025

Related U.S. Application Data
(60) Provisional application No. 63/632,288, filed on Apr. 10, 2024.

(51) Int. Cl. A61M 37/00; B81C 1/00

(52) U.S. Cl. CPC A61M 37/0015; B81C 1/00111; B81C 1/00349; A61M 2037/0053

(57) ABSTRACT

A continuously variable stacked extrusion (CVSE) process for forming a microneedle. The process includes growing a plurality of quantities of a material in a growth direction, pulling a terminal portion of the grown material, and breaking the terminal portion. Growth in the growth direction is accomplished with continuous contact between each quantity of material.

### CROSS-REFERENCE TO RELATED APPLICATION

This application claims priority to U.S. Provisional Application Ser. No. 63/632,288, filed Apr. 10, 2024, the entire content of which is incorporated herein by reference.

### FIELD

This invention relates to additive manufacturing and additive-manufactured compositions.

### BACKGROUND

Microneedles (MN) are an emerging tool in medicine and wearable sensors, with many variations and applications including dissolvable, microfluidic/wicking, and conductive types for drug delivery, interstitial fluid sampling, and in situ electrochemical sensing.

Wearable, real-time biosensors that can monitor biosignatures and biomarkers of performance, stress, health and other physiological states of the airmen and warfighters could increase performance and protect the health of individuals as well as generate larger, multi-user data sets that may offer insight about occupational conditions and tasks and human factor design. Microneedles (MNs) have emerged as one of the leading candidate technologies for monitoring human performance by sensing biomarkers in the interstitial fluid (ISF) of the skin using a minimally invasive wearable device.

The primary characteristics of MNs useful in wearables are mechanical rigidity sufficient to penetrate the skin, mechanical strength/toughness to resist breaking, adequate conductivity to maintain signal amplitude, and ability to be functionalized, and biocompatibility. Lastly, to move the technology from a laboratory prototype to a deployable system, the manufacturing of devices must be scalable, robust, and economical. Thus, the challenge is to develop a combination of materials and design that meet all these goals.

Currently, MN manufacturing methods are unreliable and difficult to scale, slowing the progress of clinical testing and development of such deployable MN products. The present disclosure addresses an unmet need for manufacturing spike-like MN constructs and/or features required for biointerfaces, wearables, and other applications. The present disclosure provides a new manufacturing methodology that is convenient and affordable with versatility in its compatibility/application to specific substrates/devices/manufacturing environment and vastly expands the shapes/profiles/designs that can be fabricated. The disclosed methodology allows more freedom in the materials and design of MN and more freedom and control over where they are placed and on what.

### SUMMARY

Continuously variable stacked extrusion (CVSE) is an additive manufacturing (AM) approach that enables creation of micrometer-sized features well below what can be conventionally achieved using extrusion-based AM such as fused filament fabrication or extrude-and-cure. CVSE is able to achieve these micro-scale resolutions by drawing viscous materials to a point spaller.

CVSE is distinguished from fused deposition modelling (FDM) or fused filament fabrication (FFF), in that the extruded form is built with continuous contact with the prior “stack”, rather than relying on discontinuous fusion of the molten thermoplastic or thermoset to the a previously printed layer.

CVSE is distinguished from drawing lithography by the ability to deposit structures anywhere within a three-dimensional build space, on a build plate, 3D surface, or bath/vat. It also allows for more complex structures by offering control of many more process variables and interdependent linking of these variables.

The methods of the present disclosure have applications to microneedles (MNs), which are an emerging tool in medicine and wearable sensing, with many variations and applications including dissolvable, microfluidic/wicking, and conductive types for drug delivery, interstitial fluid sampling, and in-situ electrochemical sensing. Additionally, CVSE may also be used to fabricate neural probes for stimulation and recording of brain, nerve or muscle activity. CVSE is especially suited to fabrication of microstructures, but can be applied to more macro objects. Additional applications are envisioned for microstructures in microfluidic devices as interfaces, supports, filters, connectors, probes for sampling injecting, stimulating and recording, etc. CVSE can be used to fabricate radially varied strings (flexible) or rods. It should be understood that the methods of the present disclosure have numerous applications that are not explicitly enumerated in the present disclosure.

In an aspect, a continuously variable stacked extrusion (CVSE) process for forming a microneedle comprises: applying a first quantity of a material having viscoelastic properties to a build plate via an extruder orifice, thereby producing a plant phase; applying a second quantity of the material to the plant phase via the extruder orifice, thereby forming a pile phase; applying at least one additional quantity of the material to the pile phase via the extruder orifice, thereby producing growth of the pile phase in a growth direction; pulling a terminal portion of the pile phase by moving the extruder orifice in the growth direction, thereby forming a variable extrusion diameter; and breaking the terminal portion of the pile from the extruder orifice thereby forming the microneedle. Growth in the growth direction is accomplished with continuous contact between each quantity of material.

In another aspect, a process for increasing surface porosity and surface area of a device comprises applying the microneedle formed in accordance with other aspects disclosed herein.

In yet another aspect, a CVSE process for forming a microneedle may include pushing a material having viscoelastic properties onto a build plate via an extruder orifice; pulling a portion of the material by moving the extruder orifice away from the build plate, thereby forming a variable extrusion diameter; and breaking the material from the extruder orifice, thereby forming the microneedle.

In an aspect, the present disclosure provides an AM approach of CVSE. The defining feature of CVSE is that the object is built upon itself in a primarily axial direction (Z) of the extrusion orifice rather than horizontally radially as is done in conventional FFF. A construct printed via CVSE is shaped by varying the rate/volume of extrusion through the height of the feature. Continuously variable stacked extrusion allows for precise modulation of a vertical feature's diameter. This may include inward and outward variation which can be used to fabricate cones, inverted cones, barbs, fins, plate stacks, spirals and indentations. The CVSE process includes two regimes: (1) the Positive Regime, where the volume of extruded material is equal to or greater than the surface area of extruder hot-end orifice multiplied by the stack/lift height; and (2) the Negative Regime, where the volume of material deposited is less than the volume defined by the extruder orifice diameter multiplied by the stack's lift height. In the extreme the volume may be zero, or even negative, which indicates retraction. Ultimately, the pointed tips required for MINs are created by a negative phase, wherein the necking and cooling of the filament leads to a break, just as in drawing lithography.

In another aspect, the present disclosure provides constructs generated using the CVSE method.

In still another aspect, the present disclosure provides an apparatus comprising constructs generated using the CVSE method.

### BRIEF DESCRIPTION OF THE DRAWINGS

The accompanying drawings, which are incorporated in and constitute a part of this specification, illustrate embodiments of the invention and, together with a general description of the invention given above, and the detailed description given below, serve to explain the invention. The patent or application file contains at least one drawing executed in color. Copies of this patent or patent application publication with color drawing(s) will be provided by the Office upon request and payment of the necessary fee.

FIGS. 1a-1g depict illustrations of a CVSE process according to the present disclosure;

FIG. 2 depicts an illustration of a CVSE process apparatus, including two exemplary nozzle orifice shapes;

FIG. 3a-3c depict exemplary illustrations of asymmetric forms, such as, e.g., leans (FIG. 3a), offsets (FIG. 3b), and helices (FIG. 3c);

FIG. 4 depicts bulged (panel a) and smooth (panel b) forms achievable by processes according to the present disclosure;

FIG. 5 depicts exemplary unique shapes including, e.g., bulge (panel a), ripple (panel b), beads (panel c), and flare (panel d), achievable by processes according to the present disclosure;

FIG. 6 is an exemplary illustration of CVSE via embedding in a support matrix;

FIG. 7 is a photograph of an exemplary result of a singular deposit and draw without CVSE stacking;

FIG. 8 shows pictures of irregular profiles with different “pull” speeds;

FIGS. 9a-9b are pictures of additional examples;

FIG. 10 is a photograph of a printer table with deposited samples on top of the surface;

FIG. 11 is a closeup photograph of an extrudate deposited on top of the surface;

FIG. 12 is a micrograph of a CVSE deposit, scale bar 500 μm, inset box surrounding the microneedle tip 30 μm, showing the needle tip is sharp with a tip likely under 5 μm;

FIG. 13 shows a prior apparatus for Z-direction pull steps of a multiplexed extrusion process of drawing lithography, which illustrates multi-point fabrication;

FIG. 14 is an exemplary biosensor product comprising a plurality of microneedles according to the present disclosure; and

FIG. 15 shows various exemplary microneedle profiles according to the present disclosure.

### DETAILED DESCRIPTION

The following Detailed Description refers to accompanying drawings to illustrate exemplary embodiments consistent with the present disclosure. References in the Detailed Description to “one embodiment,” “an embodiment,” “an exemplary embodiment,” etc., indicate that the exemplary embodiment described can include a particular feature, structure, or characteristic, but every exemplary embodiment does not necessarily include the particular feature, structure, or characteristic. Moreover, such phrases are not necessarily referring to the same embodiment. Further, when a particular feature, structure, or characteristic is described in connection with an embodiment, it is within the knowledge of those skilled in the relevant art(s) to affect such feature, structure, or characteristic in connection with other exemplary embodiments whether or not explicitly described.

The exemplary embodiments described herein are provided for illustrative purposes, and are not limiting. Other embodiments are possible, and modifications can be made to exemplary embodiments within the scope of the present disclosure. Therefore, the Detailed Description is not meant to limit the present disclosure. Rather, the scope of the present disclosure is defined only in accordance with the following claims and their equivalents.

Embodiments of the present invention may be implemented in hardware, firmware, software, or any combination thereof. Embodiments of the present invention may also be implemented as instructions stored on a machine-readable medium, which may be read and executed by one or more processors. A machine-readable medium may include any mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, and/or instructions may be described herein as performing certain actions. However, it should be appreciated that such descriptions are merely for convenience and that such actions in fact result from computing devices, processors, controllers, or other devices executing the firmware, software, routines, instructions, etc.

For purposes of this discussion, each of the various components discussed may be considered a module, and the term “module” shall be understood to include at least one of software, firmware, and hardware (such as one or more circuit, microchip, or device, or any combination thereof), and any combination thereof. In addition, it will be understood that each module may include one, or more than one, component within an actual device, and each component that forms a part of the described module may function either cooperatively or independently of any other component forming a part of the module. Conversely, multiple modules described herein may represent a single component within an actual device. Further, components within a module may be in a single device or distributed among multiple devices in a wired or wireless manner.

The following Detailed Description of the exemplary embodiments will so fully reveal the general nature of the present disclosure that others can, by applying knowledge of those skilled in the relevant art(s), readily modify and/or adapt for various applications such exemplary embodiments, without undue experimentation, without departing from the scope of the present disclosure. Therefore, such adaptations and modifications are intended to be within the meaning and plurality of equivalents of the exemplary embodiments based upon the teaching and guidance presented herein. It is to be understood that the phraseology or terminology herein is for the purpose of description and not limitation, such that the terminology or phraseology of the present specification is to be interpreted by those skilled in relevant art(s) in light of the teachings herein.

Microneedles (MNs) are an emerging platform for medicine and biomonitoring. They may be used to deliver drugs, sample interstitial fluid (ISF), or detect and measure biochemical concentrations in vivo. These different functions are facilitated by various materials, designs, and supporting platforms. Various manufacturing approaches and fabrication methods have been devised to accommodate other materials and designs. 3D printing or additive manufacturing has demonstrated potential for MN fabrication but limitations in materials and resolution exist. Material and resolution constraints of replica molding and 3D printed MN molds have limited the quality and function of efforts to use them with their electrochemical aptamer-based (EAB) biosensors.

The methods of the present disclosure has applications to microneedles (MNs), which are an emerging tool in medicine and wearable sensing, with many variations and applications including dissolvable, microfluidic/wicking, and conductive types for drug delivery, interstitial fluid sampling, and in-situ electrochemical sensing. Additionally, CVSE may also be used to fabricate neural probes for stimulation and recording of brain, nerve or muscle activity. CVSE is especially suited to fabrication of microstructures, but can be applied to more macro objects. Additional applications are envisioned for microstructures in microfluidic devices as interfaces, supports, filters, connectors, probes for sampling injecting, stimulating and recording, etc. CVSE can be used to fabricate radially varied strings (flexible) or rods. This manufacturing process also allows for simple and reliable fabrication of non-monotonic structures, where the radius is not solely decreasing with the height of the structure, but may increase or decrease. There are many functions afforded by non-monotonic shapes. Conventional molding and even centrifugal-drawing (to create “tissue-interlocking” microneedles) are limited in their ability to fabricate non-monotonic shapes. Molding has difficulty getting the material in place and removing it fully without breakage. Centrifugal drawing lithograph requires multiple steps and does not provide such control over the radial profile afforded by the use of positive and negative phases of CVSE. It should be understood that the methods of the present disclosure have numerous applications that are not explicitly enumerated in the present disclosure.

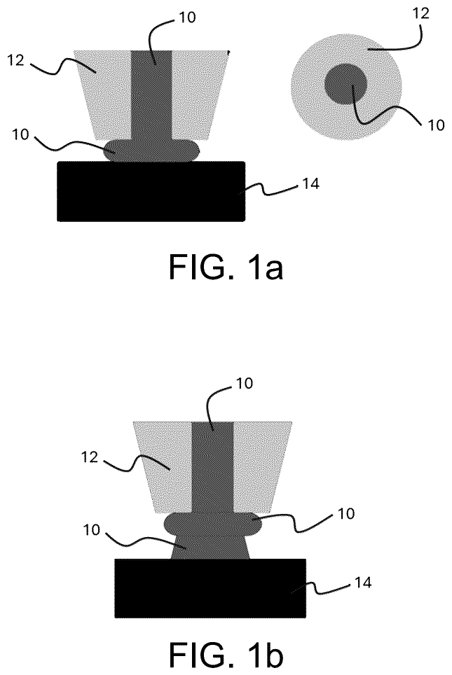

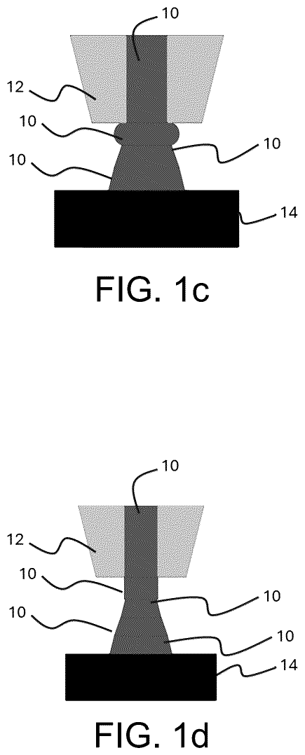

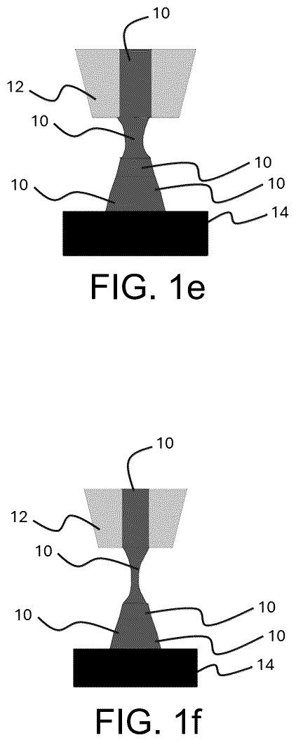

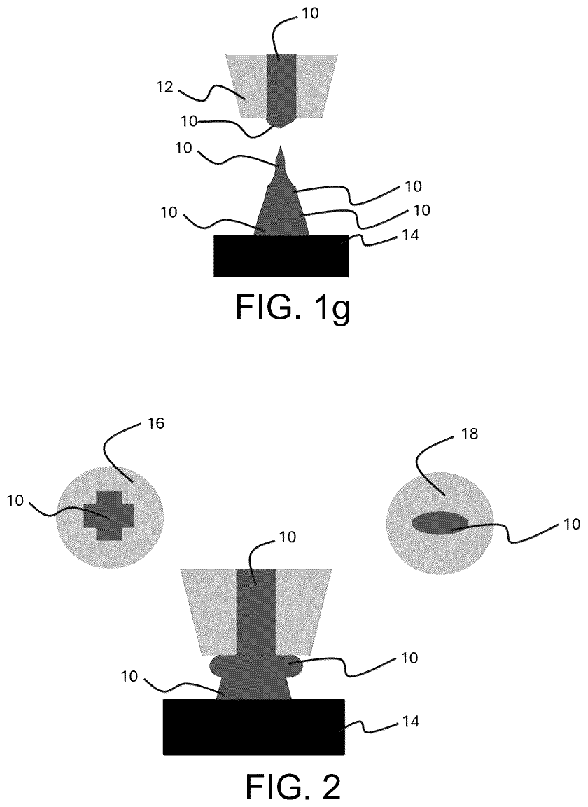

In an aspect, the present disclosure provides an AM approach of CVSE. The defining feature of CVSE is that the object is built upon itself in a primarily axial direction (Z) of the extrusion orifice rather than horizontally radially as is done in conventional FFF (see FIGS. 1a-1g). A construct printed via CVSE is shaped by varying the rate/volume of extrusion through the height of the feature. Continuously variable stacked extrusion allows for precise modulation of a vertical feature's diameter. This may include inward and outward variation which can be used to fabricate, e.g., cones, inverted cones, barbs, fins, plate stacks, spirals, and indentations.

Turning now to FIG. 1a-1g, an exemplary CVSE process includes depositing extruded material 10 through an extruder “hot-end” orifice 12 onto build plate 14. A further quantity of extruded material 10 is then applied atop the original extruded material 10 (best shown in FIG. 1b). Subsequent extruded materials 10 may then be applied sequentially atop the preceding extruded materials 10. Each quantity of extruded material deposited may be referred to as a “stack,” which will have a volume, height, and other typical dimensions.

The CVSE process includes two regimes: (1) the Positive Regime, where the volume of extruded material 10 is equal to or greater than the surface area of extruder hot-end orifice 12 multiplied by the stack/lift height (best shown in FIG. 1a); and (2) the Negative Regime, where the volume of material deposited is less than the volume defined by the hot-end orifice 12 diameter multiplied by the stack's lift height (best shown in FIG. 1d and FIG. 1e). In the extreme, the volume may be zero, or even negative, indicating retraction. Ultimately, the pointed tips required for MNs are created by a negative phase, wherein the necking and cooling of the filament leads to a break just as in drawing lithography (best shown in FIG. if and FIG. 1g).

In conventional additive manufacturing vertical/z-axis movements are used to change layers, by changing the net distance of the extrusion nozzle/focal plane from the build surface (for fused filament fabrication or photopolymerizable resins respectively). Whereas, horizontal (X-axis and Y-axis) movements are used to deposit a 2D slice of the 3D object. More exotic non-planar/conformal printing still print a single surface layer, though it may be deformed along a non-planar surface.

CVSE is distinguished from fused deposition modelling (FDM) or fused filament fabrication (FFF), in that the extruded form is built with continuous contact with the prior “stack,” rather than relying on discontinuous fusion of the molten thermoplastic or thermoset to the previously printed layer. This distinction produces a significant difference in the structures formed.

This does not exclude the attachment of the CVSE-based construct being attached by extruding onto a construct printed via standard AM, even with the same print head. In fact, though specialized nozzles can extend the functionality CVSE, it is possible to do with standard FFF thermoplastic hot ends and nozzles.

Despite the quantized steps of most translation stages and electric motors, “continuous” refers to the ability to vary the extrusion rate/volume with resolution beyond typical ‘layer-heights’ of other AM techniques. Even with a lower limit dictated by a single step of a stepper motor translation system, greater resolution/continuity can be afforded due to the physical continuity of nozzle pressures that have a time dependence (lag) between quantized steps. However, CVSE as a process does not exclude abrupt changes like steps if desired, these are simply “variable stacked extrusion” that still maintains continuous contact between slices of the stack.

The unavoidable quantization of movement intrinsic to stepping motors also affects the minimum extrudable volume (MEV, found by multiplying the extrusion length per E-step by the cross-sectional area of the filament). This represents the smallest theoretical volume of material that the printer can reliably extrude, calculated from the minimum E-step the stepper motor can take. One ‘mode’ of CVSE then determines each stack height (distance the print head moves away from the prior stack) so that the stack volume (stack height multiplied by stack diameter) is an integer multiple of the MEV.

This method of determining stack height may also be applied to negative or drawing phase. In this case the volume of any ‘draw’ (which may also be “stacked” or have multiple operations performed sequentially, where (temporally) adjacent draw the end and start at the same point.) In this case, rather than calculate volumes using a rectangle rule (diameter times height) the volume will be calculated more precisely with an integration of the function describing the desired NIN profile. Such functions are dependent on the dynamic viscoelastic properties of the material which is undergoing phase change due to controllable parameters including nozzle temperature, air temperature (which can be controlled down or up, for example by cooling fan or infrared (IR) laser respectively) and interfacial properties between the material and the nozzle (charge, hydrophobicity).

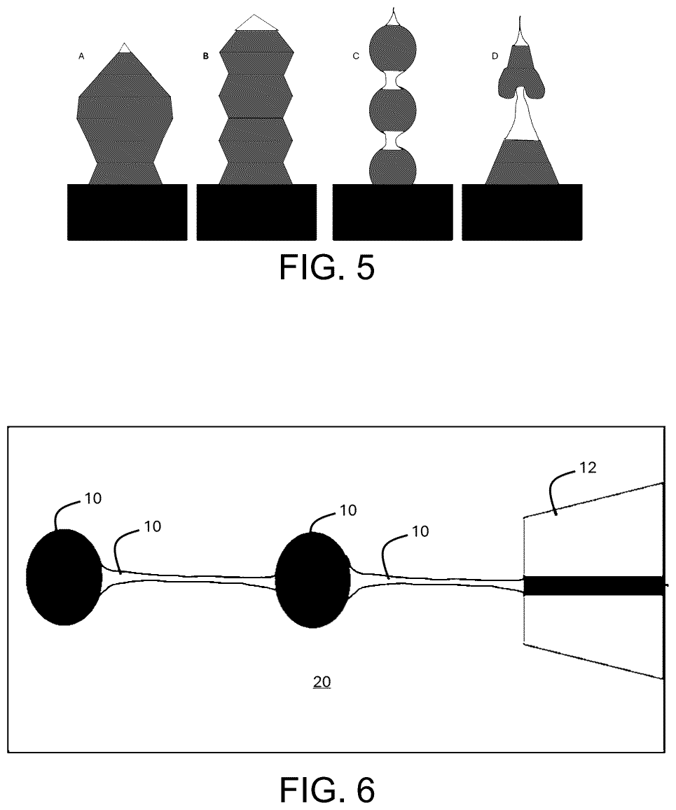

CVSE is distinguished from drawing lithograph in its more precise control over the ‘tapering” of a drawn construct. Because CVSE uses consecutive stacking, it can create more complex profiles and it can increase the diameter of a construct with height, leading to unique shapes (FIG. 5) with many new functionalities.

“Stacking” refers to the deposition of material between two boundaries. The far boundary may initially include a print surface or bed or viscous bath or the prior extruded slice in the stack and the near boundary includes the extrusion orifice, filled with extrudable material, and the nozzle surface surrounding the orifice, as well as any potential solid, flexible or viscous materials that limit by contact force, the direction in which material can expand, such that stack diameter, surface area (non-cylindrical) expands radially (perpendicular to vectors normal to both boundary surfaces).

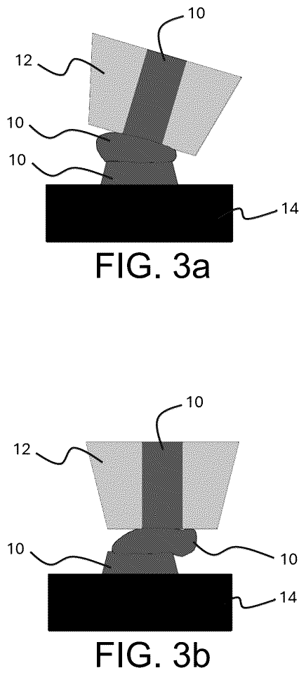

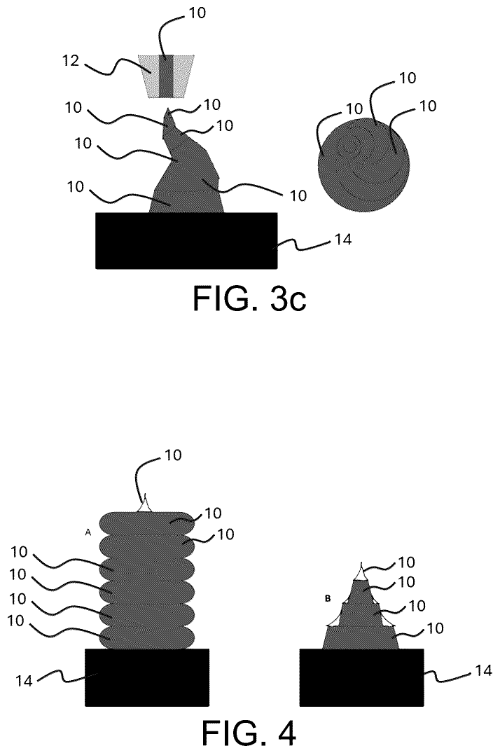

CVSE may be implemented solely with vertical movement to produce axially symmetric (as defined by the nozzle orifice shape) constructs such as microneedles, posts, barbs, and cups. FIG. 2 shows two different extruder orifice shapes, an “X” 16 and an oval 18. Non-circular nozzle orifices can be used with CVSE to print non-axially symmetric constructs (such as bars with variable rectangular cross-sections). However, CVSE is not strictly confined to axial/vertical movement and may include small movements in X-axis, Y-axis and nozzle angle. These secondary movements allow for the printing of axially asymmetric forms like screws or and bends, as shown in FIGS. 3a-3c. In such an implementation the construct is still produced with continuous, vertically connected extrusion rather than discontinuous layers.

While CVSE is a continuous process, multiple CVSE actions can be chained together and the CVSE process itself has two regimes related to extrusion rates and extrusion diameters relative to the nozzle orifice. In the pushing or Positive Regime, the ratio of the filament extrusion rate to the nozzle lift speed is such that the diameter of the construct exceeds the diameter of the orifice. In this regime, the extrusion rate is always positive, and printed material is squeezed between the trailing material and the nozzle surface and may even extend (bulge) beyond those surfaces. In the drawing or Negative Regime, the ratio of extrusion rate to lift speed is such that “necking” occurs leading to a decrease in diameter. This regime may include extrusion rates that are positive (but is sufficient to maintain a diameter of trailing material, zero, or negative (retraction).

Even in the Positive Regime, the transition between each “stack” is not necessarily discontinuous marked by an angular interface between stacks. In fact, to achieve a stacked bulge, as shown in panel a of FIG. 4, with crevices between bulges, sufficient cooling is required to keep the prior stack from melding into the current stack due to the effects of surface tension and viscosity. This is the standard scenario in FFF where the extruder hot-end is moved away from prior layers allowing them to completely solidified before an additional layer is deposited on top. This can also be done in CVSE to create bulges or even flares, as shown in panel a of FIG. 5. However, in the basic implementation of CVSE, the extruder hot-end orifice 12 is always in contact with the extruded material 10, and therefore the prior layer may not completely solidify. This can be leveraged to create smooth transitions between stacks, reducing or eliminating crevices and edges at their stack interface, as shown in panel b of FIG. 4.

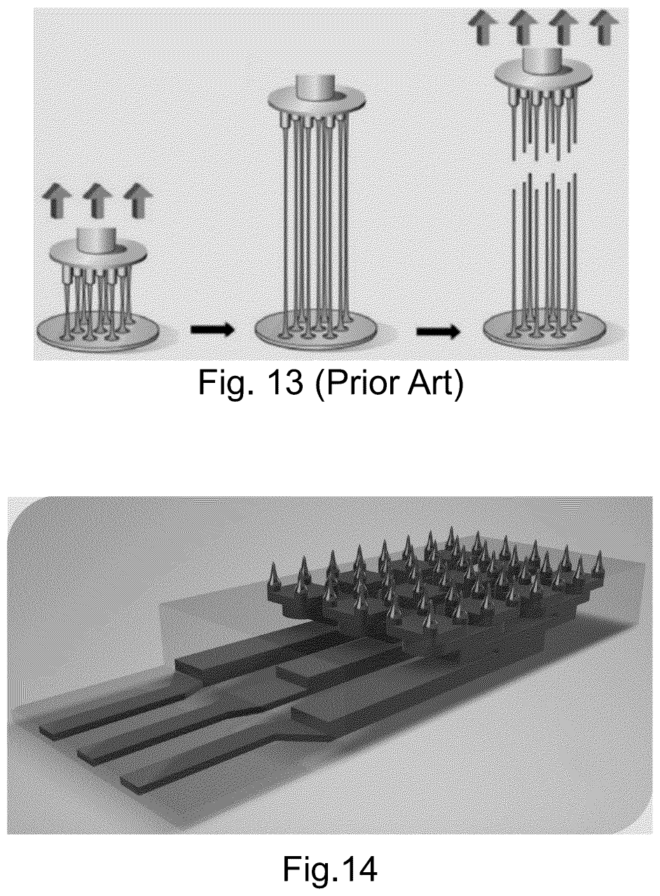

The diameter of any stack or phase may not only decrease in relation to the prior stack. They may also increase to create bulbs, bulges (FIG. 5, panel a), ripples (FIG. 5, panel b), or beads (FIG. 5, panel c). By reversing the Z-axis motion after a negative or drawn operation, flares can even be created (FIG. 5, panel d). These shapes/profiles can be used to help anchor microneedles, increase the surface area of probes or supports for chemical reactions, and support filtration/separation through a forest of structures. Forests of complex profiles pictured in FIG. 5 may additionally be used to increase porosity when used as scaffolds. (See also FIG. 13). Many profiles may be produced in accordance with the process disclosed herein, as shown in FIG. 15, which is intended to be exemplary and in no way limiting of the subject matter disclosed herein.

The CVSE process can be applied to thermoplastics and thermosets or other phase transition materials such, and may be photoinitiated, chemically crosslinked, or cured by other means, depending upon the material used. In the case of other materials besides thermoplastics, the extruder orifice may be a syringe tip, for example. Exemplary non-thermoplastic materials include, but are not limited to, UV-curables and other phase-change materials, where the phase change may be magnetic property phase changes, for example. In embodiments, superparamagnetic nano rods could result in a flowable to solid transition)

CVSE has several advantages over conventional microfabrication approaches. It is more versatile in the materials it can use and the variety of shapes it can produce. It allows for precise fabrication of structures (e.g., microneedles) onto electrode pads, enabling a solution to interconnect problems. It is cheaper to perform than conventional microfabrication. In relation to implantable microelectrode arrays as neural probes, these often require significant etching steps.

In an aspect, the present disclosure provides compositions manufactured according the CVSE process of the present disclosure.

The compositions may comprise microneedles (MNs).

The compositions may be radially symmetric or radially asymmetric. The compositions may have a “blade-like” radial isotropy, which may be formed by tilting the extrusion orifice, or by a preconfigured orifice shape. The compositions are not confined to the initial build surface. Longer constructs can be built by ‘grabbing’ cured/cooled/already printed stacks at points closer to the nozzle/extrusion operation, allowing parts of the construct on the other side of the grab/clamp to be coiled, rolled, or otherwise collected.

During extrusion, extrusion diameter may be controlled by a quantized extrusion volume calculated from a desired stack layer height and desired stack cross-sectional area.

The variable extrusion diameter may be determined by necking due to viscoelastic flow of extruded material with a dynamic viscosity effected by temperature, crosslinking degree, surface energy/tension.

The crosslinking degree may be controlled by photoinitiated crosslinking, bath-based chemical crosslinking, pH change physical crosslinking, vapor based crosslinking, or electromagnetic field control of phase (through nanoparticle alignment), which is in turn controlled by electromagnetic intensity/energy density, exposure time, total energy applied.

Each step of the stack may include not only an extrusion/deposition, but also a retraction to eliminate ‘bulging’ or introduce concavity to individual disks of thickens dz.

Constructs, including but not limited to microneedles, with complex defined radial cross sections, may be created from combining a CVSE phase with a drawing/necking phase during which a desired disk diameter is no longer purely achieved by extruding a volume of filament equal to the volume of the desired disk, but that previously extruded material, or under extrusion is combined with vertical lift to produce necking of the thermoplastic, facilitating construct diameters less than the nozzle ID. The necking phase may be controlled via modification of temperature, draw speed, cooling intensity, UV intensity, or some other mode that causes a rheological change in the material.

An imaging device may be used to capture the profile of the extruded construct in either CVSE or necking phase, and this image is used for real time feedback control of parameters and machine learning is used to generate a model allowing for generation of extrusion parameters/protocol from a design/curve of the construct.

The nozzle surface/charge may be dynamically controlled to change properties besides temperature, such as charge, hydrophobicity/hydrophilicity, in order to control wetting/adhesion of the extruded materials to the nozzle. This can be used to improve control and uniformity of the extruded constructs especially in the drawing phase.

As shown in FIG. 6, the extruded material 10 may be extruded into a bath/vat containing another material which acts as a support matrix 20, allowing for the creation of stacks that are not self-supporting. Without such a support matrix 20, gravity necessitates a mostly vertical stack.

A controlled atmosphere, bath, or support matrix may be temperature controlled, which allows for prolonged cooling times which can allow for more extreme necking and profiles when using CVSE with thermoplastics.

A vapor chamber, bath or support matrix may contain a chemical crosslinker for the extruded material, allowing for solidification/phase change beyond thermoplastics.

The extruded material may be extruded in a microgravity environment.

The extrusion may include coaxial extrusion of other materials, including fugitive materials, including gasses and liquids to form hollows, or materials with different properties that add mechanical support of other functionalities (electrical or thermal conductivity, magnetic materials, diffracting materials and so on). These materials can be extruded with rates/volumes (or with ‘disk’ diameters) that push the outer shell(s) material(s) out radially. The fugitive material may be controlled not by a rate or volume but with a pressure.

Stack heights may be set so that the stack volume is an integer multiple of the minimum extrusion volume (MEV). Stack heights may be calculated based on an MEV, with the additional consideration for the ‘final’ stack and/or ‘drawn’ feature. For example, MNs may have very sharp and thin tips, which have/require a volume less than the MEV. To achieve sub-MEV volumes in the final stack/feature, the ‘extra’ material is ‘shifted’ or extruded in prior stacks, especially bottom stacks where variations in volume are less significant. In this way, the MN is built from the bottom up, but the extrusion and lift values for each stack/phase of the MN are calculated from the top (tip) down.

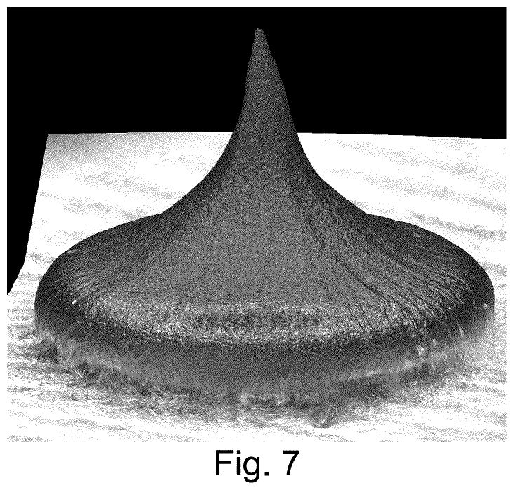

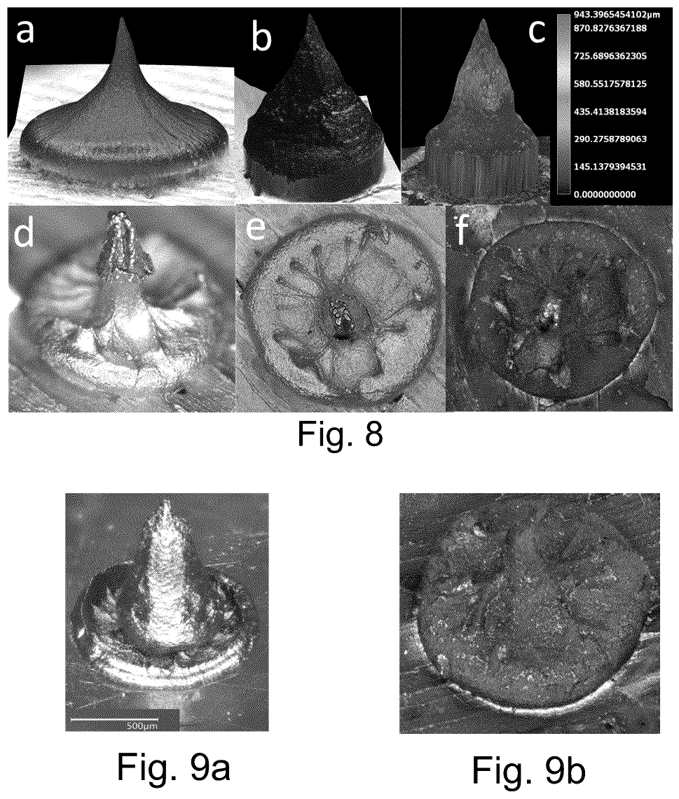

Various profiles for the constructs produced are possible via modifications to the protocols. For example, FIG. 7 is a photograph of an exemplary result of a singular deposit and draw without CVSE stacking. FIG. 8 provides pictures of irregular profiles with different “pull” speeds. FIGS. 9a-9b are pictures of additional examples.

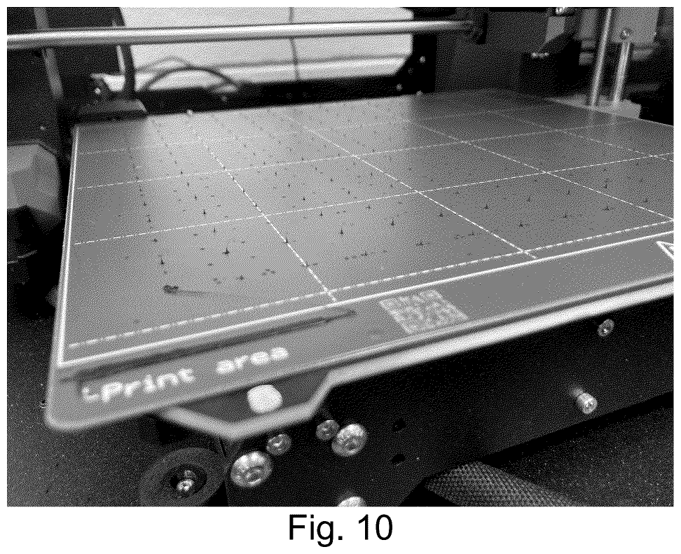

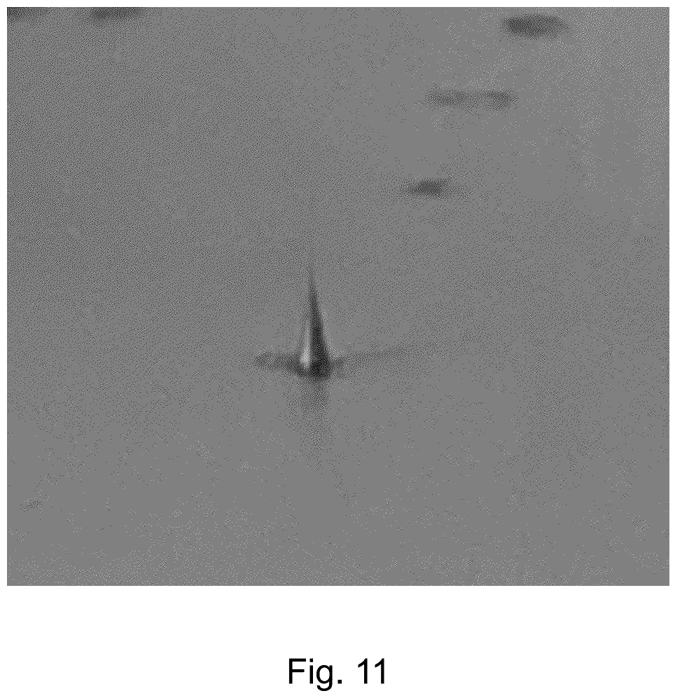

To illustrate the scale of the materials that may be produced by the process described herein, FIG. 10 is a photograph of a printer table with deposited samples on top of the surface. FIG. 11 is a closeup photograph of an extrudate deposited on top of the surface.

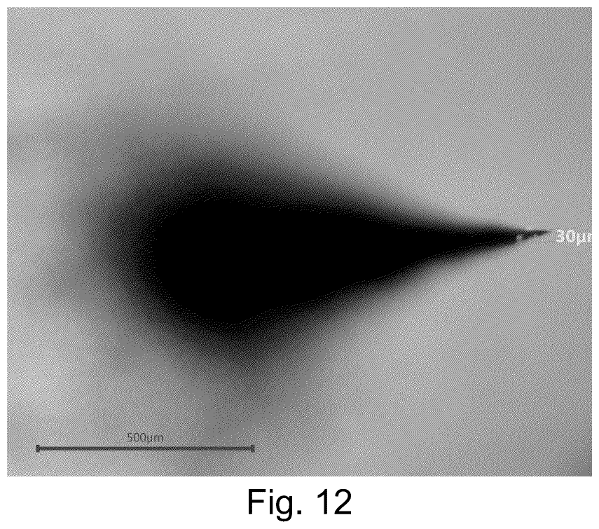

FIG. 12 is a micrograph of a CVSE deposit, scale bar 500 μm. As shown in FIG. 12, the needle tip is sharp with a tip likely under 5 μm.

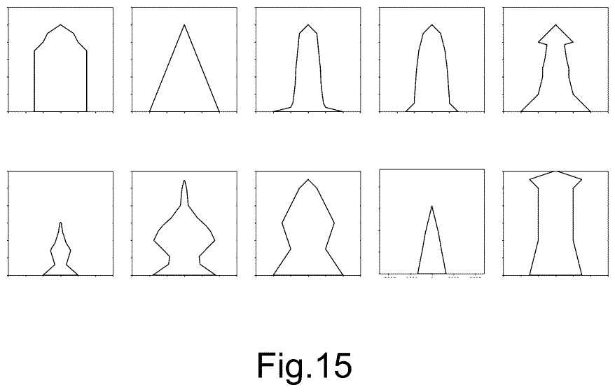

In embodiments, a plurality of extruded products 10 may be produced simultaneously. FIG. 13 shows a prior apparatus for Z-direction pull steps of a multiplexed extrusion process of drawing lithography, which illustrates multi-point fabrication, i.e., not using the extruder orifices of the presently disclosed subject matter. FIG. 14 is an exemplary biosensor product comprising a plurality of microneedles produced in accordance with a multiplexed version of the presently disclosed subject matter.

Complex CVSE protocol recipes may be used to create structures with complex axial profiles, such as but not limited to, bulbs, ripples, beads, and flares. Such structures, and arrays and forests of such structures, may be used for anchoring, increasing surface area and porosity. ‘Flares’ and/or ‘barbs’ may be used to assist in holding a microneedle (or patch thereof) in place.

In summary, the processes disclosed herein include two regimes: a push regime, and a pull regime. In the push regime, viscoelastic material is extruded between the nozzle orifice and a “solid” material (build plate, cooled/solidified previously extruded material) such that a compressive stress is applied between the nozzle and the extruded material. As material is extruded, the resulting pressure within the viscoelastic material between the nozzle and the “solid” is relieved by radial expansion material. This push mode is effective for forming slices of the stack with diameters greater than that of the nozzle orifice. In the pull regime, beginning with viscoelastic material extruded between the nozzle and the ‘solid,” the distance between the nozzle and the solid is increased, resulting in tensile stress, with a resulting decrease in the radius of the viscoelastic material via necking. This pull mode is effective for forming slices of the stack with diameters less than the nozzle orifice. Coordinating the pull mode with the phase-change of the viscoelastic material towards a solid is effectively applied to ‘break’ the strained material and produce pointed tips with various cone angles.

As described above, the CVSE process may include a plant phase, one or more pile phases, one or more pull phases, and break phase. The plant phase is a push regime that includes applying a first quantity of material having viscoelastic properties to a build surface via an extruder orifice to create a base or anchor. The pile phase is a push regime that includes applying an additional, variable quantity of material onto the solidified ‘backing’ material extruded in the prior phase to produce (add) a ‘slice’ to the stack with diameter greater than the diameter of the nozzle orifice, dependent on the backing-nozzle distance and the volume of material extruded. Pile phases can be added repeatedly. The pull phase is a pull regime that includes applying additional viscoelastic material between the nozzle and backing such that the cleft is bridged by the material. While the material is still viscoelastic, the distance between the nozzle and the backing is increased in a controlled manner, resulting in a necking of the material. As the rate of change of backing-nozzle distance and the viscoelasticity of the material are varied in concert, the cone angle of the ‘slice’ can be controlled. The break phase is a pull regime that is a ‘final’ phase in which continuity is broken between the material attached to the backing and that connected to the nozzle. As the rate of change of backing-nozzle distance and the viscoelasticity of the material varies in concert, a solid tip can be formed. Any number of pile and pull phases can be repeated and combined between the plant and break phases. Additionally, multiple sequences starting with plant and ending with break phases can be combined for even more process flexibility.

### Clauses

1. A continuously variable stacked extrusion (CVSE) process for forming a microneedle, the process comprising:
applying a first quantity of a material having viscoelastic properties to a build plate via an extruder orifice in a push regime, thereby producing a plant phase;
applying a second quantity of the material to the plant phase via the extruder orifice, thereby forming a pile phase;
applying at least one additional quantity of the material to the pile phase via the extruder orifice, thereby producing growth of the pile phase in a growth direction;
pulling a terminal portion of the pile phase by moving the extruder orifice in the growth direction, thereby forming a variable extrusion diameter; and
breaking the terminal portion of the pile from the extruder orifice thereby forming the microneedle;
wherein growth in the growth direction is accomplished with continuous contact between each quantity of material.

2. The process according to clause 1, further comprising tilting of the extrusion orifice, thereby growing the microneedles without radial symmetry.

3. The process according to clause 1 or clause 2, wherein the variable extrusion diameter is controlled by a quantized extrusion volume calculated from a desired stack layer height and cross-sectional area.

4. The process according to any one of clauses 1-3, wherein the variable extrusion diameter is determined by necking due to viscoelastic flow of extruded material with a dynamic viscosity effected by temperature, crosslinking degree, and surface energy/tension.

5. The process according to clause 4, wherein the crosslinking degree is controlled by photoinitiated crosslinking, which is in turn controlled by electromagnetic intensity/energy density, exposure time, and total energy applied.

6. The process according to any one of clauses 1-5, wherein the applying the first quantity of material, the applying the second quantity of the material, and the applying the at least one additional quantity of the material, further comprises retracting the extruder orifice.

7. The process according to any one of clauses 1-6, further comprising at least one additional cycle of the applying at least one additional quantity of the material and the pulling the terminal portion prior to the breaking the terminal portion.

8. The process according to any one of clauses 1-7, wherein the variable extrusion diameter is controlled via modification of temperature, draw speed, cooling intensity, UV intensity, or any process variable that causes a rheological change in the material.

9. The process according to any one of clauses 1-8, wherein an imaging device is used to capture a profile of at least one of the plant phase, the pile phase, the growth of the pile phase, the variable extrusion diameter, and the microneedle.

10. The process according to clause 9, wherein the profile is used for real-time feedback control of parameters.

11. The process according to clause 10, further comprising using machine learning to generate a model allowing for generation of extrusion parameters, a protocol, or both extrusion parameters and a protocol, from the profile.

12. The process according to any one of clauses 1-11, further comprising dynamically controlling a change of the extrusion orifice or a change of the build plate such that wetting, adhesion, or wetting and adhesion of the material to the extruder orifice is promoted.

13. The process according to any one of clauses 1-12, wherein the microneedle is formed in a bath comprising a support matrix material.

14. The process according to clause 13, wherein the bath or the support matrix material is temperature controlled.

15. The process according to clause 13, wherein the bath or the support matrix contains a chemical crosslinker complementary to the material.

16. The process according to any one of clauses 1-15, wherein the process takes place in a microgravity environment.

17. The process according to any one of clauses 1-16, wherein the microneedle is not confined to the build plate.

18. The process according to any one of clauses 1-17, further comprising coaxially extruding at least one other material.

19. The process according to clause 18, wherein the at least one other material comprises a fugitive material.

20. The process according to clause 19, wherein the fugitive material comprises a gas, a liquid, or a gas and a liquid.

21. The process according to clause 18, wherein the at least one other material is applied to radially expand the plant phase, the pile phase, or the plant phase and the pile phase.

22. The process according to clause 19, wherein an amount of the fugitive material is controlled via a pressure.

23. The process according to any one of clauses 1-22, wherein a height of the plant phase, the pile phase, or the plant phase and the pile phase, are set so that a volume of material is an integer multiple of a minimum extrusion volume (MEV).

24. The process according to clause 23, wherein the height of the plant phase, the pile phase, or the plant phase and the pile phase, is calculated based on the MEV and the terminal portion comprises a volume less than the MEV.

25. The process according to any one of clauses 1-24, wherein the microneedle comprises a complex axial profile.

26. The process according to clause 25, wherein the complex axial profile comprises a bulb, a ripple, a bead, or a flare.

27. A process for increasing surface porosity and surface area of a device, said process comprising applying the microneedle formed in accordance with clause 1 to the device.

28. The process according to clause 27, wherein an array or a forest of a plurality of the microneedles is applied to the device.

29. The process according to clause 28, wherein each of the plurality of the microneedles is held in place with a flare or a barb.

30. A continuously variable stacked extrusion (CVSE) process for forming a microneedle, the process comprising:
pushing a material having viscoelastic properties onto a build plate via an extruder orifice;
pulling a portion of the material by moving the extruder orifice away from the build plate, thereby forming a variable extrusion diameter; and breaking the material from the extruder orifice, thereby forming the microneedle.

### Example

Examples related to the present disclosure are described below. In most cases, alternative techniques can be used. The examples are intended to be illustrative and are not limiting or restrictive of the scope of the invention as set forth in the claims.

Continuously Variable Stacked Extrusion is an approach to generating optimized G-code for microneedle fabrication. Below are the theoretical foundations and implementation details of the volume-first slicing strategy, which ensures precise material control while maintaining geometric accuracy.

### Core Concepts

Minimum Extrusion Volume (MEV): The fundamental unit of this approach is the Minimum Extrusion Volume (MEV). This represents the smallest volume of material that the printer can reliably extrude, calculated from the minimum E-step the stepper motor can take. Every slice created must contain a whole number of MEVs to ensure consistent material flow.

Z-Step Quantization: While ideal positions may be calculated mathematically, the printer can only move in discrete steps determined by its mechanical properties. This creates a fundamental tension between where the optimal position of the extruder orifice is and where it can actually be positioned. This tension is controlled through error tracking and compensation.

Volume-First Philosophy: Rather than starting with fixed slice heights and trying to match volumes to them, this process uses a volume-first approach. First, the volume that would give a whole number of MEVs is determined. Then, the position that achieves this volume while respecting the printer's mechanical constraints is determined.

### The CVSE Process

1. Profile Analysis: before slicing begins, the target profile coordinates are loaded, the cap height is calculated (where profile radius equals nozzle radius), special interpolation strategies are used to address edge cases, and start and end positions are determined based on plant mode

2. Slice Generation Process: for each slice, the following steps are performed.

Initial Setup: Start at the current Z position (initially the cap height). Determine processing mode (max resolution or standard). Calculate target thickness based on previous error and mode. Establish thickness bounds (MIN_SLICE_THICKNESS to 2.5× target)

Position Evaluation (Max Resolution Mode): Calculate exact Z position for exactly 1 MEV. Attempt multiple search strategies to find the optimal position. Quantize to printer steps (floor and ceiling). Choose the position giving volume closest to 1 MEV.

Position Evaluation (Standard Mode): Generate a range of candidate MEV counts based on slice thickness. For thicker slices (>100 μm), use a wider range up to 2× ideal count. For thinner slices, use a focused search near ideal MEV count. Find the MEV count that gives thickness closest to target. Determine the Z position that gives exactly this volume.

Error Calculation and Tracking: Calculate the exact Z that would give perfect volume (unquantized). Calculate height error as difference between quantized and exact Z. Store error for compensating future slices. Apply different compensation factors based on slice location. For the top region, use a 0.25× error compensation factor (reduced effect). For other regions, use a 0.5× error compensation factor (standard effect)

Slice Creation and Validation: Create slice with calculated parameters. Validate all slice properties (position, volume, thickness). Add to slice collection. Update Z position for next slice.

3. Volume Calculations: The volume calculations handle the cone-like geometry of each slice. Volume=(π×h/3)×(R1²+R2²+R1×R2), where: h=slice height in mm, R1=upper radius in mm, and R2=lower radius in mm. The implementation includes boundary validation and coordinate conversion. Thus, this process clamps Z values to valid profile range to prevent errors, which addresses interpolation of radii at arbitrary Z positions and converts between microns (internal) and mm³ (volume units).

4. Error Handling and Compensation: two types of errors are tracked, volume error and height error. Volume error addresses how far the actual volume is from a whole number of MEVs. Height error addresses how far the quantized position is from the ideal position. Volume tolerance is determined dynamically as Volume Tolerance=π×(filament_diameter/2)²×0.0001. This represents the volume change from a 0.1 micron variation in E-step. Height errors are accumulated and used to adjust subsequent slice thicknesses with a reduced compensation (0.25×) near the needle tip, standard compensation (0.5×) for the rest of the profile, and a bounded adjustment to maintain slice thickness constraints.

### Error Compensation in CVSE Processing

Understanding Z-axis Error Compensation: In CVSE printing, the relationship between Z-movement and material extrusion is direct and precise. Each slice represents a specific volume of material that must be distributed over an exact vertical distance. When the Z-axis movements are quantized to mechanical step sizes, a robust error compensation strategy may be needed to maintain geometric accuracy.

The Physical Reality of Layer Formation: When the material is extruded during printing, two critical factors determine the final geometry, the volume of material extruded (controlled by E-steps), and the vertical distance over which that material is distributed (Z-movement). These factors are inextricably linked. If the Z-movement differs from the calculated ideal position due to step quantization, the same volume of material gets distributed over a different height than intended. This creates an immediate discrepancy between the actual and intended positions.

Immediate Error Compensation Strategy: An immediate, full error compensation, rather than gradual correction across multiple layers, is used. This approach is based on the physical nature of the printing process. Consider a simple example, with target slice thickness of 50 microns, an actual movement due to step quantization of 52 microns. A volume of material extruded is calculated for 50 microns. As a result, material intended for 50 microns is spread over 52 microns, such that the extruded layer is 2 microns higher than intended. The next slice must fully compensate for this error by being 2 microns thinner. That is, the next target thickness is 48 microns (50−2). The material volume is calculated for 48 microns, which results in a return to the intended Z-position

This immediate compensation is superior to gradual correction for several reasons. Layer Interdependence: Each layer's material distribution directly affects its adjacent layers. The thickness of one layer immediately impacts the starting position of the next. Error Locality: By correcting errors immediately, positional discrepancies do not propagate beyond adjacent layers, thus maintaining the overall geometric accuracy of the profile. Material Distribution: The sooner the return to the intended Z-positions, the more accurately the designed material distribution throughout the profile is maintained. Profile Accuracy: Immediate correction helps maintain the intended profile geometry by minimizing the duration and extent of any deviations from the target shape.

### Implementation Details

The code to implement this strategy allows for careful error tracking and compensation. The error compensation is bounded only by the minimum and maximum slice thickness constraints.

### Error Compensation Workflow

Position Selection: Find best available Z-position (quantized to printer steps). Calculate actual thickness achieved. Determine difference from target thickness.

Error Calculation: Compute full position error. Store for next slice compensation. Log error metrics for monitoring.

Next Slice Adjustment: Apply full error compensation to target thickness. Validate adjusted thickness against bounds. Calculate new target volume based on adjusted thickness.

Validation: Ensure adjusted thickness meets minimum requirements. Verify volume constraints are satisfied. Confirm printer mechanical limits are respected.

This approach ensures that the printed structure maintains the highest possible geometric accuracy while respecting the mechanical constraints of the printing system.

Error Recovery Strategy: When errors occur, detailed error information is logged, safe fallback values are determined, and processing is continued if possible. Slice generation is discontinued if error is unrecoverable.

It is to be appreciated that the Detailed Description section, and not the Abstract section, is intended to be used to interpret the claims. The Abstract section can set forth one or more, but not all exemplary embodiments, of the present disclosure, and thus, is not intended to limit the present disclosure and the appended claims in any way.

While the present invention has been illustrated by the description of one or more embodiments thereof, and while the embodiments have been described in considerable detail, they are not intended to restrict or in any way limit the scope of the appended claims to such detail. Additional advantages and modifications will readily appear to those skilled in art. The invention in its broader aspects is therefore not limited to the specific details, representative apparatus and method and illustrative examples shown and described. Accordingly, departures may be made from such details without departing from the scope of the general inventive concept.

1. A continuously variable stacked extrusion (CVSE) process for forming a microneedle, the process comprising:
applying a first quantity of a material having viscoelastic properties to a build plate via an extruder orifice in a push regime, thereby producing a plant phase;
applying a second quantity of the material to the plant phase via the extruder orifice, thereby forming a pile phase;
applying at least one additional quantity of the material to the pile phase via the extruder orifice, thereby producing growth of the pile phase in a growth direction;
pulling a terminal portion of the pile phase by moving the extruder orifice in the growth direction, thereby forming a variable extrusion diameter; and
breaking the terminal portion of the pile from the extruder orifice thereby forming the microneedle;
wherein growth in the growth direction is accomplished with continuous contact between each quantity of material.

2. The process according to claim 1, further comprising tilting of the extrusion orifice, thereby growing the microneedles without radial symmetry.

3. The process according to claim 1, wherein the variable extrusion diameter is controlled by a quantized extrusion volume calculated from a desired stack layer height and cross-sectional area.

4. The process according to claim 1, wherein the variable extrusion diameter is determined by necking due to viscoelastic flow of extruded material with a dynamic viscosity effected by temperature, crosslinking degree, and surface energy/tension.

5. The process according to claim 4, wherein the crosslinking degree is controlled by photoinitiated crosslinking, which is in turn controlled by electromagnetic intensity/energy density, exposure time, and total energy applied.

6. The process according to claim 1, wherein the applying the first quantity of material, the applying the second quantity of the material, and the applying the at least one additional quantity of the material, further comprises retracting the extruder orifice.

7. The process according to claim 1, further comprising at least one additional cycle of the applying at least one additional quantity of the material and the pulling the terminal portion prior to the breaking the terminal portion.

8. The process according to claim 1, wherein the variable extrusion diameter is controlled via modification of temperature, draw speed, cooling intensity, UV intensity, or any process variable that causes a rheological change in the material.

9. The process according to claim 1, wherein an imaging device is used to capture a profile of at least one of the plant phase, the pile phase, the growth of the pile phase, the variable extrusion diameter, and the microneedle.

10. The process according to claim 9, wherein the profile is used for real-time feedback control of parameters.

11. The process according to claim 10, further comprising using machine learning to generate a model allowing for generation of extrusion parameters, a protocol, or both extrusion parameters and a protocol, from the profile.

12. The process according to claim 1, further comprising dynamically controlling a change of the extrusion orifice or a change of the build plate such that wetting, adhesion, or wetting and adhesion of the material to the extruder orifice is promoted.

13. The process according to claim 1, wherein the microneedle is formed in a bath comprising a support matrix material.

14. The process according to claim 13, wherein the bath or the support matrix material is temperature controlled.

15. The process according to claim 13, wherein the bath or the support matrix contains a chemical crosslinker complementary to the material.

16. The process according to claim 1, wherein the process takes place in a microgravity environment.

17. The process according to claim 1, wherein the microneedle is not confined to the build plate.

18. The process according to claim 1, further comprising coaxially extruding at least one other material.

19. The process according to claim 18, wherein the at least one other material comprises a fugitive material.

20. The process according to claim 19, wherein the fugitive material comprises a gas, a liquid, or a gas and a liquid.

21. The process according to claim 18, wherein the at least one other material is applied to radially expand the plant phase, the pile phase, or the plant phase and the pile phase.

22. The process according to claim 19, wherein an amount of the fugitive material is controlled via a pressure.

23. The process according to claim 1, wherein a height of the plant phase, the pile phase, or the plant phase and the pile phase, are set so that a volume of material is an integer multiple of a minimum extrusion volume (MEV).

24. The process according to claim 23, wherein the height of the plant phase, the pile phase, or the plant phase and the pile phase, is calculated based on the MEV and the terminal portion comprises a volume less than the MEV.

25. The process according to claim 1, wherein the microneedle comprises a complex axial profile.

26. The process according to claim 25, wherein the complex axial profile comprises a bulb, a ripple, a bead, or a flare.

27. A process for increasing surface porosity and surface area of a device, said process comprising applying the microneedle formed in accordance with claim 1 to the device.

28. The process according to claim 27, wherein an array or a forest of a plurality of the microneedles is applied to the device.

29. The process according to claim 28, wherein each of the plurality of the microneedles is held in place with a flare or a barb.

30. A continuously variable stacked extrusion (CVSE) process for forming a microneedle, the process comprising:
pushing a material having viscoelastic properties onto a build plate via an extruder orifice;
pulling a portion of the material by moving the extruder orifice away from the build plate, thereby forming a variable extrusion diameter; and
breaking the material from the extruder orifice, thereby forming the microneedle.
